SEQUENCE LISTING (20 sequences)

SEQ ID NO: 1 - DNA, 19 bp, synthetic construct
  misc_feature 1..19: note = Forward primer for qPCR of the gene mTNF-alpha
  source 1..19: mol_type = other DNA
caggcggtgcctatgtctc

SEQ ID NO: 2 - DNA, 22 bp, synthetic construct
  misc_feature 1..22: note = Reverse primer for qPCR of the gene mTNF-alpha
  source 1..22: mol_type = other DNA
cgatcaccccgaagttcagtag

SEQ ID NO: 3 - DNA, 21 bp, synthetic construct
  misc_feature 1..21: note = Forward primer for qPCR of the gene mIL-6
  source 1..21: mol_type = other DNA
ttggtccttagccactcctcc

SEQ ID NO: 4 - DNA, 23 bp, synthetic construct
  misc_feature 1..23: note = Reverse primer for qPCR of the gene mIL-6
  source 1..23: mol_type = other DNA
tagtccttcctaccccaatttcc

SEQ ID NO: 5 - DNA, 22 bp, synthetic construct
  misc_feature 1..22: note = Forward primer for qPCR of the gene mIL-1
  source 1..22: mol_type = other DNA
gcaactgttcctgaactcaact

SEQ ID NO: 6 - DNA, 21 bp, synthetic construct
  misc_feature 1..21: note = Reverse primer for qPCR of the gene mIL-1
  source 1..21: mol_type = other DNA
atcttttggggtccgtcaact

SEQ ID NO: 7 - DNA, 20 bp, synthetic construct
  misc_feature 1..20: note = Forward primer for qPCR of the gene mCOX-2
  source 1..20: mol_type = other DNA
tgagtggggtgatgagcaac

SEQ ID NO: 8 - DNA, 20 bp, synthetic construct
  misc_feature 1..20: note = Reverse primer for qPCR of the gene mCOX-2
  source 1..20: mol_type = other DNA
ttcagaggcaatgcggttct

SEQ ID NO: 9 - DNA, 22 bp, synthetic construct
  misc_feature 1..22: note = Forward primer for qPCR of the gene mEGFR
  source 1..22: mol_type = other DNA
ggagagaatccctttggagaac

SEQ ID NO: 10 - DNA, 22 bp, synthetic construct
  misc_feature 1..22: note = Reverse primer for qPCR of the gene mEGFR
  source 1..22: mol_type = other DNA
ccagttctgtttgtcccatagt

SEQ ID NO: 11 - DNA, 23 bp, synthetic construct
  misc_feature 1..23: note = Forward primer for qPCR of the gene mINOS
  source 1..23: mol_type = other DNA
gttctcagcccaacaatacaaga

SEQ ID NO: 12 - DNA, 19 bp, synthetic construct
  misc_feature 1..19: note = Reverse primer for qPCR of the gene mINOS
  source 1..19: mol_type = other DNA
gtggacgggtcgatgtcac

SEQ ID NO: 13 - DNA, 20 bp, synthetic construct
  misc_feature 1..20: note = Forward primer for qPCR of the gene mcatenin
  source 1..20: mol_type = other DNA
ggcaaccctgaggaagaaga

SEQ ID NO: 14 - DNA, 20 bp, synthetic construct
  misc_feature 1..20: note = Reverse primer for qPCR of the gene mcatenin
  source 1..20: mol_type = other DNA
gggatgagcagcgtcaaact

SEQ ID NO: 15 - DNA, 20 bp, synthetic construct
  misc_feature 1..20: note = Forward primer for qPCR of the gene mMyc
  source 1..20: mol_type = other DNA
ggaacgtcagaggaggaacg

SEQ ID NO: 16 - DNA, 20 bp, synthetic construct
  misc_feature 1..20: note = Reverse primer for qPCR of the gene mMyc
  source 1..20: mol_type = other DNA
tgctcgtctgcttgaatgga

SEQ ID NO: 17 - DNA, 18 bp, synthetic construct
  misc_feature 1..18: note = Forward primer for qPCR of the gene mCyclinD1
  source 1..18: mol_type = other DNA
tcctgctaccgcacaacg

SEQ ID NO: 18 - DNA, 19 bp, synthetic construct
  misc_feature 1..19: note = Reverse primer for qPCR of the gene mCyclinD1
  source 1..19: mol_type = other DNA
gaccagcctcttcctccac

SEQ ID NO: 19 - DNA, 20 bp, synthetic construct
  misc_feature 1..20: note = Forward primer for qPCR of the gene m-actin
  source 1..20: mol_type = other DNA
ggctgtattcccctccatcg

SEQ ID NO: 20 - DNA, 22 bp, synthetic construct
  misc_feature 1..22: note = Reverse primer for qPCR of the gene m-actin
  source 1..22: mol_type = other DNA
ccagttggtaacaatgccatgt

(19) United States
(12) Patent Application Publication
(10) Pub. No.: US 2026/0014225 A1
(43) Pub. Date: Jan. 15, 2026

(54) USE OF HULLESS BARLEY GRASS EXTRACT IN CONTROLLING COLORECTAL CANCER AND ULCERATIVE COLITIS

(71) Applicants: Shanghai Jiao Tong University, Shanghai (CN); Shanghai Ganya Biotechnology Co., Ltd., Shanghai (CN); Shanghai Aifa Investment Management Co., Ltd., Shanghai (CN)

(72) Inventors: Zhongquan SUI, Shanghai (CN); Liu NAN, Shanghai (CN); Mengting MA, Shanghai (CN); Sheng NAN, Shanghai (CN); Xiao NAN, Shanghai (CN); Mingming WANG, Shanghai (CN); Zhiqian WU, Shanghai (CN); Zijun LI, Shanghai (CN)

(73) Assignees: Shanghai Jiao Tong University, Shanghai (CN); Shanghai Ganya Biotechnology Co., Ltd., Shanghai (CN); Shanghai Aifa Investment Management Co., Ltd., Shanghai (CN)

(21) Appl. No.: 18/999,098

(22) Filed: Dec. 23, 2024

(30) Foreign Application Priority Data
Jul. 15, 2024 (CN) 202410944366.8

(51) Int. Cl. A61K 36/8998; A61P 1/04; A61P 35/00; B01D 11/02; B01D 15/20; B01J 20/285; B01J 20/30

(52) U.S. Cl. CPC A61K 36/8998; A61P 1/04; A61P 35/00; B01D 11/0265; B01D 11/0288; B01D 15/20; B01J 20/285; B01J 20/3071; A61K 2236/333; A61K 2236/51; A61K 2236/53; A61K 2236/55; B01J 2220/52

(57) ABSTRACT

Disclosed is the use of a hulless barley grass extract containing polyphenol in controlling colorectal cancer and ulcerative colitis. In terms of the ulcerative colitis, the drug ameliorates a weight loss of colitis mice, reduces an oxidative stress level of colitis mice, reduces relative mRNA expression levels of inflammatory factors TNF-α, IL-6, and IL-1β, and effectively improves a pathological damage caused by inflammatory response in the colon tissue of ulcerative colitis model mice. In terms of the colorectal cancer, the drug is shown to prevent and/or inhibit the occurrence and development of ulcerative colitis and associated colorectal cancer thereof by inhibiting the overexpression of a Wnt signaling pathway and reducing the inflammatory response and oxidative stress damage. The raw materials of the extract are derived from natural products, which are easy to collect and highly safe.

### CROSS-REFERENCE TO RELATED APPLICATION

This patent application claims the benefit and priority of Chinese Patent Application No. 202410944366.8 filed with the China National Intellectual Property Administration on Jul. 15, 2024, the disclosure of which is incorporated by reference herein in its entirety as part of the present application.

### REFERENCE TO SEQUENCE LISTING

A computer readable XML file entitled “GWP20240805834_sequence listing”, which was created on Oct. 31, 2024, with a file size of about 26,652 bytes, contains the sequence listing for this application, has been filed with this application, and is hereby incorporated by reference in its entirety.

### TECHNICAL FIELD

The present disclosure relates to the technical field of biomedicine, and relates to the use of a hulless barley grass extract in controlling colorectal cancer and ulcerative colitis. The present disclosure specifically relates to a preparation method of a hulless barley grass-derived polyphenol extract and use thereof in controlling ulcerative colitis and associated colorectal cancer thereof.

### BACKGROUND

According to data from the World Health Organization (WTO), cancer deaths accounted for ⅙ of the total number of deaths in the world in 2020. Among the new cancer cases in the world in 2020, colorectal cancer ranked third and was the most deadly cancer next only to lung cancer. By 2025, the number of cancer patients may be expected to rise to 25 million. Currently, surgical resection is generally used for stage 0 to stage II colorectal cancer, and chemotherapy drugs such as 5-FU, folinic acid, and oxaliplatin are generally used for stage II colorectal cancer. However, the development of tumor cell resistance currently limits the anti-tumor effect of chemotherapy drugs. Patients with stage III and stage IV colorectal cancer are generally treated with a combination of chemotherapy and targeted therapy. According to the molecular pathogenic pathway and genetic mechanism, colitis-associated colorectal cancer can be identified as a colitis-associated disease, and long-term ulcerative colitis can develop into colorectal cancer. A study has shown that the incidence of colitis-associated colorectal cancer is directly related to the severity and duration of the inflammatory response during the patient's illness, and probability of cancer is more than 1.7 times that of healthy people. The specific mechanism is to promote the occurrence of colorectal cancer through an inflammation-dysplasia-cancer cascade reaction, accompanied by oxidative stress damage and DNA double-strand breaks. Another animal experiment on the correlation between colitis and colorectal cancer has shown that a single injection of carcinogens in mice with colitis can lead to multiple colon tumors, while mice without inflammation require multiple injections of carcinogens and a longer treatment time to form tumors. Therefore, it may have a positive significance in reducing the early inflammatory response of colitis-associated colorectal cancer to reduce the risk of ulcerative colitis, thereby preventing and inhibiting the occurrence and development of cancer.

In the ongoing search for safer and more effective methods for the prevention and treatment of cancer, natural compounds derived from plants have attracted great interest due to their advantages such as no or almost no adverse reactions, high bioavailability, high accuracy, and safe mode of action. In the ongoing research on natural products, people have found that some terpenes, alkaloids, sterols, polysaccharides, and polyphenols in plants are effective anti-cancer compounds. In vitro observations, as well as preclinical and epidemiological studies have shown that long-term use of these compounds can prevent and inhibit the occurrence and development of tumors. Most of these natural plant compounds with functional effects cannot be synthesized in the human body, such that diet is the main source of such compounds for human beings. Colitis-associated colorectal cancer is a common cancer with a high mortality rate. Since this cancer mainly occurs in the colon and has a long disease cycle, it is more likely to be prevented and inhibited by consuming natural plant compounds.

Hulless barley, also known as naked barley, has the characteristics of strong cold resistance, short growth period, high yield, early development, and wide adaptability. Southwest China, as a main barley-producing area in China, has the lowest cancer mortality rate, of which the Tibet region shows the lowest incidence of colorectal cancer in China. This may be closely related to hulless barley, the staple food in this region. The hulless barley grass is about 20 cm to 30 cm in length. Such leaves are the seedlings from ten days after the germination of hulless barley to a heading stage. At this time, the hulless barley reaches a nutritional peak before its reproductive cycle. Several studies have shown that hulless barley grass has certain therapeutic or auxiliary therapeutic effects on cancer. Compared with that of barley leaves, a high-ultraviolet-radiation, cold, and drought environment in which hulless barley grass grows causes changes in the composition and content of more than 100 secondary metabolites, including phenolic acids, flavonoids, triterpenes, polysaccharides, proanthocyanidins, and chlorophyll. However, there are no patent literature reports on the research of hulless barley grass extract in controlling ulcerative colitis and associated colorectal cancer thereof at home and abroad.

### SUMMARY

In view of the defects in the prior art, an objective of the present disclosure is to provide the use of a hulless barley grass extract in controlling colorectal cancer and ulcerative colitis. In the present disclosure, the preventive and therapeutic effects of hulless barley grass-derived polyphenol extract on ulcerative colitis and associated colorectal cancer thereof are discovered for the first time, and the effectiveness is verified by a colitis mouse model and a colon cancer mouse model. Moreover, compared with the hulless barley grass-derived polysaccharide extract and lipid extract, as well as hulless barley seed-derived polyphenol extract in the prior art, the polyphenol extract has a more significant effect, providing a new idea and means for the treatment of ulcerative colitis and associated colorectal cancer thereof.

Specifically, the hulless barley grass-derived polyphenol extract can effectively improve the survival rate and colon length of mice with acute ulcerative colitis. The drug ameliorates the weight loss of colitis mice, reduces an oxidative stress level of colitis mice, reduces relative mRNA expression levels of inflammatory factors TNF-α, IL-6, and IL-1β, and effectively improves pathological damage caused by inflammatory response in the colon tissue of ulcerative colitis model mice. Furthermore, the hulless barley grass-derived polyphenol extract significantly reduces the colon weight, tumor number, tumor size, and serum IL-6 level of mice with colorectal cancer, and leads to significant downregulation of gene expression for the TNF-α, IL-6, IL-1β, COX-2, EGFR, and INOS in colon tissue; the hulless barley grass-derived polyphenol extract can also downregulate the mRNA expression level of the genes related to a Wnt signaling pathway.

In the present disclosure, active substances are extracted from hulless barley grass as raw materials, which are economic, environmental-friendly, highly safe, low-cost, and easily available. The active ingredients derived from hulless barley grass have desirable development and application prospects in the preparation of a drug for controlling ulcerative colitis and associated colorectal cancer, thus greatly improving the economic value of the hulless barley grass. On one hand, the extract will be of great significance to the search for novel anti-colorectal cancer drugs and the prevention of colorectal cancer; on the other hand, the extract will play a positive role in promoting the industrialization of high-quality and healthy hulless barley-based foods, thus improving the relatively backward economic conditions in plateau areas.

The objective of the present disclosure can be achieved by the following technical solutions.

In a first aspect, the present disclosure provides the use of a hulless barley grass-derived polyphenol extract in preparation of a drug for preventing and/or treating ulcerative colitis and associated colorectal cancer thereof, where
a preparation method of the hulless barley grass-derived polyphenol extract includes the following steps:
S1, subjecting a hulless barley grass whole powder to alcohol extraction, evaporation, and freeze-drying to obtain a crude polyphenol extract; and
S2, separating and purifying the crude polyphenol extract using a chromatography column loaded with an activated saturated wet resin to obtain the hulless barley grass-derived polyphenol extract.

In an embodiment, the alcohol extraction in step S1 includes: mixing the hulless barley grass whole powder with a methanol solution to allow an ultrasonic treatment and centrifugation in sequence to obtain a supernatant.

Further, the methanol solution has a mass percentage of 70% to 90%.

Further, the hulless barley grass whole powder and the methanol solution are at a material-to-liquid ratio of 1 g:(20-25) mL.

Further, the ultrasonic treatment is conducted under a power of 480 W to 560 W at 40° C. to 50° C. for 30 min to 60 min.

Further, a residual precipitate after centrifugation is mixed with methanol and ultrasonically extracted 2 to 3 times.

In an embodiment, in step S1, the evaporation is conducted at 40° C. to 70° C.; and the freeze-drying is conducted at −50° C. to −100° C.

In some preferred embodiments, the evaporation is conducted using a rotary evaporator at 55° C.; and freeze-drying is conducted at −80° C.

In an embodiment, a preparation process of the activated saturated wet resin in step S2 includes: immersing a macroporous resin in an ethanol solution, washing with deionized water, immersing in a hydrochloric acid solution and a sodium hydroxide solution, and then adjusting a pH value in sequence.

Further, the macroporous resin includes an AB-8 macroporous resin.

Further, the macroporous resin is immersed in an ethanol solution with a mass percentage of 80% to 95% for 15 h to 28 h.

Further, the washing is conducted until there is no white floating matter and no alcohol smell.

Further, the total volume of the hydrochloric acid solution and the sodium hydroxide solution is 3 to 5 times the volume of the macroporous resin; and the macroporous resin is immersed in a hydrochloric acid solution with a mass percentage of 3% to 5% and a sodium hydroxide solution with a mass percentage of 2% to 5% for 2 h to 6 h.

Further, the pH is adjusted to neutral by washing with deionized water.

In some preferred embodiments, a preparation process for the activated saturated wet resin includes: immersing the macroporous resin in a 95% ethanol solution for 24 h, and washing with deionized water until there is no white floating matter and no alcohol smell. The resin was immersed in 5% hydrochloric acid solution and 2% sodium hydroxide solution three times a volume of the resin for 4 h, and then washed with deionized water until neutral.

In an embodiment, the separating in step S2 includes dissolving the crude polyphenol extract into a sample solution, loading the sample solution onto the chromatography column, removing impurities, and then eluting the hulless barley grass-derived polyphenol extract to obtain an eluate.

In an embodiment, the sample solution has a concentration of 2 mg/mL to 3 mg/mL, 4 BV of the sample solution is loaded at a flow rate of 2 BV/min to 3 BV/min, the impurities are removed with 4 BV of deionized water, and then the hulless barley grass-derived polyphenol extract is eluted with 70% ethanol at a flow rate of 2.5 BV/min to 3 BV/min.

In an embodiment, the purifying in step S2 includes evaporating and vacuum freeze-drying the separated eluate.

Further, the vacuum freezing is conducted at −30° C. to −40° C. for 24 h to 48 h.

In some preferred embodiments, a separation process is to wet-load the activated saturated wet resin into a chromatography column, dissolve the crude extract of phenolic substances in hulless barley grass with 80% methanol to prepare a sample solution, load the sample solution, wash with deionized water to remove impurities, elute a target substance with 70% ethanol, collect the eluate, remove the ethanol by rotary evaporation, and vacuum freeze-dry the solid to obtain a freeze-dried powder of the hulless barley grass-derived polyphenol extract.

Compared with the prior art, the present disclosure has the following beneficial effects:

1. The present disclosure has discovered new use for hulless barley grass-derived polyphenol extract.

2. In the present disclosure, it is found through animal model experiments that the hulless barley grass-derived polyphenol extract can effectively improve the survival rate and colon length of mice with acute ulcerative colitis. The drug ameliorates the weight loss of colitis mice, reduces an oxidative stress level of colitis mice, reduces relative mRNA expression levels of inflammatory factors TNF-α, IL-6, and IL-1β, and effectively improves pathological damage caused by inflammatory response in the colon tissue of ulcerative colitis model mice.

3. In the present disclosure, it is found through animal model experiments that the hulless barley grass-derived polyphenol extract significantly reduces the colon weight, tumor number, tumor size, and serum IL-6 level of mice with colorectal cancer, and leads to significant downregulation of gene expression for the TNF-α, IL-6, IL-1β, COX-2, EGFR, and INOS in colon tissue; the hulless barley grass-derived polyphenol extract can also downregulate the mRNA expression level of genes related to Wnt signaling pathway. In summary, the hulless barley grass-derived polyphenol extract plays a role in preventing and/or inhibiting the occurrence and development of colorectal cancer by inhibiting the overexpression of the Wnt signaling pathway and reducing the inflammatory response and oxidative stress damage in the early stage of colorectal cancer.

4. Compared with the hulless barley grass-derived polysaccharide extract and lipid extract as well as hulless barley seed-derived polyphenol extract in the prior art, the hulless barley grass-derived polyphenol extract is more effective in preventing and/or inhibiting the occurrence and development of colorectal cancer.

5. In the present disclosure, the raw materials come from natural products, which are not only easy to collect but also highly safe. The method has a simple extraction process and low cost and is suitable for industrial production and market promotion.

### BRIEF DESCRIPTION OF THE DRAWINGS

Other features, objectives, and advantages of the present disclosure will become more apparent by reading the detailed description of non-limiting embodiments with reference to the following accompanying drawings.

FIG. 1 shows the process of a mouse acute ulcerative colitis modeling experiment;

FIG. 2 shows the process of a mouse colorectal cancer modeling experiment;

FIG. 3A-FIG. 3C show the physiological status of colitis mice; where FIG. 3A is the body mass index curve of mice; FIG. 3B is the body mass index on Day 18; and FIG. 3C is DAI;

FIG. 4A-FIG. 4B show the colon length of colitis mice; where FIG. 4A is the colon length; and FIG. 4B is a typical mouse colon image;

FIG. 5A-FIG. 5B show the pathological damage on the colon tissue of colitis mice; where FIG. 5A is the H&E staining image: a is the blank group; b is the model group; c is the positive group; d is the grass polyphenol group; e is the grass polysaccharide group; f is the grass lipid group; g is the seed polyphenol group; FIG. 5B is the histological score;

FIG. 6A-FIG. 6C show the contents of GSH (FIG. 6A), LPO (FIG. 6B), and MPO (FIG. 6C) in the colon tissues of colitis mice;

FIG. 7A-FIG. 7C show the gene expression of inflammatory factors in the colon tissue of colitis mice; where (FIG. 7A) is TNF-α; (FIG. 7B) is IL-6; and (FIG. 7C) is IL-1β;

FIG. 8A-FIG. 8D show the α diversity analysis of the intestinal flora of colitis mice; where (FIG. 8A) is the Chao1 index; (FIG. 8B) is the ACE index; (FIG. 8C) is the Shannon index; and (FIG. 8D) is the rank abundance curve;

FIG. 9A-FIG. 9B show a bar graph of species distribution of intestinal flora in colitis mice; where (FIG. 9A) is the genus level; (FIG. 9B) is the phylum level;

FIG. 10A-FIG. 10B show the body weight of mice with colorectal cancer; where (FIG. 10A) is the body weight curve of mice; (FIG. 10B) is the body weight of mice on the last day;

FIG. 11A-FIG. 11C show the colon status of colorectal cancer mice; where (FIG. 11A) is the colon length; (FIG. 11B) is the colon weight; and (FIG. 11C) is the colon weight per unit length;

FIG. 12A-FIG. 12C show the tumor conditions of mice with colon cancer; where (FIG. 12A) is a typical colon picture; (FIG. 12B) is the number of tumor nodules; (FIG. 12C) is the proportion of tumors of different grades;

FIG. 13A-FIG. 13G show H&E staining images of intestinal tissues of mice with colorectal cancer; where FIG. 13A is a blank group; FIG. 13B is a model group; FIG. 13C is a positive group; FIG. 13D is a grass polyphenol group; FIG. 13E is a grass polysaccharide group;

FIG. 13F is a grass lipid group; FIG. 13G is a seed polyphenol group;

FIG. 14A-FIG. 14C show the gene expression of inflammatory factors in colorectal cancer mice; where (FIG. 14A) is TNF-α; (FIG. 14B) is IL-6; and (FIG. 14C) is IL-1β;

FIG. 15A-FIG. 15C show the expression levels of COX-2, EGFR, and INOS genes in colorectal cancer mice; where (FIG. 15A) is COX-2; (FIG. 15B) is EGFR; (FIG. 15C) is INOS;

FIG. 16A-FIG. 16D show the α diversity analysis of the intestinal flora of colorectal cancer mice; where (FIG. 16A) is the Chao1 index; (FIG. 16B) is the ACE index; (FIG. 16C) is the Shannon index; and (FIG. 16D) is the rank abundance curve;

FIG. 17 shows a bar graph of species distribution of the intestinal flora of colorectal cancer mice at the genus level;

FIG. 18A-FIG. 18C show the influence of hulless barley grass-derived polyphenol extract on the expression level of related genes in the Wnt signaling pathway of colorectal cancer mice; where (FIG. 18A) is β-catenin; (FIG. 18B) is c-Myc; and (FIG. 18C) is CyclinD1; and

FIG. 19 shows a liquid phase detection diagram of the hulless barley grass-derived polyphenol extract of Example 1.

### DETAILED DESCRIPTION OF THE EMBODIMENTS

The present disclosure will be described in detail below with reference to the drawings and specific examples. The following examples are implemented on the premise of the technical solution of the present disclosure and provide detailed implementation modes and specific operating processes, which will help those skilled in the art to further understand the present disclosure. It should be pointed out that the protection scope of the present disclosure is not limited to the following examples. Those modifications and improvements made based on the concept of the present disclosure shall all belong to the protection scope of the present disclosure.

### Example 1

This example provided the use of a hulless barley grass-derived polyphenol extract in controlling colitis-associated colorectal cancer, where a preparation method of the hulless barley grass-derived polyphenol extract included the following steps:

A certain mass of hulless barley grass whole powder was weighed, added into 80% methanol solution at a ratio of 1 g:20 mL, treated with 480 W ultrasound at 45° C. for 30 min, then centrifuged at 3,000×g for 5 min, and a supernatant was collected. The residue was extracted 2 times using the same method, and supernatants from the 3 extractions were combined and most of the methanol was removed by rotating under reduced pressure at 55° C. using a rotary evaporator to obtain a crude extract of phenolic substances from hulless barley grass. The crude extract was pre-frozen in a −80° C. refrigerator and then placed in a freeze dryer for freeze-drying to obtain a crude extract of phenolic substances derived from hulless barley grass.

AB-8 macroporous resin was immersed in 95% ethanol for 24 h and washed with deionized water until there was no white floating matter and no alcohol smell. The resin was immersed in 5% HCl and 2% sodium hydroxide in a volume three times the resin for 4 h, and the resin was washed with deionized water until it was neutral to achieve activation of the macroporous resin. The pretreated AB-8 saturated wet resin was wet loaded into a chromatography column, and 4 BV of the crude extract of phenolic substances from hulless barley grass was loaded at a concentration of 2 mg/mL and a flow rate of 2 BV/min. 4 BV of deionized water was loaded to remove most of the impurities, and then a target compound was eluted with 70% ethanol at a flow rate of 2.5 BV/min. The collected 6 BV eluate was subjected to rotation evaporation under reduced pressure at 55° C. on a rotary evaporator to remove ethanol, and then freeze-dried in a vacuum freezer to obtain a purified hulless barley grass-derived polyphenol extract. The hulless barley grass-derived polyphenol extract was stored in a dark, dry, and low-temperature place for later use in the activity evaluation of controlling ulcerative colitis.

The main chemical components of hulless barley grass-derived polyphenol extract were qualitatively analyzed by UPLC-Q-TOF-MS. As shown in FIG. 19, the main components of the hulless barley grass-derived polyphenol extract included: acacetin, robinin-7-glucoside, apigenin-4′-O-rhamnoside, apigenin-7-glucoside, apigenin-7-rutinoside, astragalin, baicalin 6-O-glucoside, oroxin B, chrysin-8-C-glucoside, chrysin 6-C-glucoside 8-C-arabinoside, chrysoeriol-7-O-glucoside, daidzein-4′,7-diglucoside, daidzin-glucoside, daidzin, puerarin, aromadendrin-3-O-arabinoside, epicatechin-3-glucoside, genistein-4′-rhamnoside, genistein-7-glucoside, genkwanin, glycitein, hesperidin-7-O-glucoside, isorhamnetin-3-O-quercetin, isorhamnetin-3-rutinoside, narcissin, kaempferol-7-glucoside, kaempferol-3-O-galactoside, kaempferol-7-O-glucoside, luteolin-7-galactoside, naringenin-7-O-glucuronide, puerarin, quercetin-3-O-neohesperidin, quercetin-3-O-robibioside, quercetin-3-O-rutinoside, quercetin-7-rutinoside, rhoifolin, rutin, scoparin, taxifolin-3-arabinoside, and tricin. The combined action of these substances made Example 1 have a desirable effect of relieving and treating ulcerative colitis and associated colon cancer thereof.

### Comparative Example 1

This comparative example provided the use of a hulless barley grass-derived polyphenol extract in controlling colitis-associated colorectal cancer, where a preparation method of the hulless barley grass-derived polyphenol extract included the following steps:

A certain mass of hulless barley grass powder was weighed and added into deionized water at a mass ratio of 1:30. A certain amount of calcium chloride was added to a resulting mixed solution such that a calcium ion concentration was (50-80) mg/L, and a pH value was adjusted to around 6. A certain amount of thermostable a-amylase was added to the mixed solution to allow enzymatic hydrolysis at 80° C. for 30 min. After the enzymatic hydrolysis, the enzyme was inactivated by placing the mixture in a water bath at 100° C. for 10 min. When the solution was cooled to 60° C., a certain amount of neutral papain was added and the mixture was incubated in a water bath for 2 h, the inactivation was repeated. When the solution was cooled to 60° C., a certain amount of saccharifying enzyme was added and the mixture was incubated in a water bath for 1 h, the inactivation was repeated. The obtained mixed solution was placed in a water bath at 80° C. for 3 h using magnetic stirring. After the water bath was completed, the residue was removed by vacuum filtration, and the obtained supernatant was concentrated in a water bath at 80° C. Under stirring, anhydrous ethanol three times the volume of the concentrate was added dropwise through a constant flow pump to produce a white precipitate. The solution was allowed to stand overnight, and then centrifuged at 3,000×g for 10 min to collect the precipitate. The hulless barley grass-derived polysaccharide extract was obtained by removing moisture through vacuum freeze-drying. The hulless barley grass-derived polysaccharide extract was stored in a dark, dry, and low-temperature place for later use in the activity evaluation of controlling ulcerative colitis.

### Comparative Example 2

This comparative example provided the use of a hulless barley grass-derived polyphenol extract in controlling colitis-associated colorectal cancer, where a preparation method of the hulless barley grass-derived polyphenol extract included the following steps:

A certain mass of hulless barley grass powder was weighed and added into petroleum ether (30° C. to 60° C.) at a mass ratio of 1:20. An obtained mixed solution was reacted in a glass reactor at 55° C. for 1 h, extracted at 65° C. for 2 h, and recovered at 75° C. for 15 min. After cooling, the mixed solution was collected and the residue was removed by suction filtration. The supernatant was placed in a rotary evaporator and the petroleum ether was removed at 60° C. to obtain the hulless barley grass-derived lipid extract. The hulless barley grass-derived lipid extract was stored in a dark, dry, and low-temperature place for later use in the activity evaluation of controlling ulcerative colitis.

### Comparative Example 3

This comparative example provided use of a hulless barley grass-derived polyphenol extract in controlling colitis-associated colorectal cancer, where the method included the following steps:

A certain mass of hulless barley seeds were ground and sieved to obtain a hulless barley powder, which was added into 80% methanol solution at a ratio of 1 g:20 mL, treated with 480 W ultrasound at 45° C. for 30 min, then centrifuged at 3,000×g for 5 min, and a supernatant was collected. The residue was extracted 2 times using the same method, and supernatants from the 3 extractions were combined and most of the acetone was removed by rotatory evaporation under reduced pressure at 55° C. using a rotary evaporator to obtain a crude extract of phenolic substances from hulless barley seeds. The crude extract was pre-frozen in a −80° C. refrigerator and then placed in a freeze dryer for freeze-drying to obtain a crude extract of phenolic substances derived from hulless barley seeds.

AB-8 macroporous resin was immersed in 95% ethanol for 24 h and washed with deionized water until there was no white floating matter and no alcohol smell. The resin was immersed in 5% HCl and 2% sodium hydroxide in a volume three times the resin for 4 h, and the resin was washed with deionized water until it was neutral to achieve activation of the macroporous resin. The pretreated AB-8 saturated wet resin was wet loaded into a chromatography column, and 4 BV of the crude extract of phenolic substances from hulless barley seeds was loaded at a concentration of 2 mg/mL and a flow rate of 2 BV/min. 4 BV of deionized water was loaded to remove most impurities, and then a target compound was eluted with 70% ethanol at a flow rate of 2.5 BV/min. The collected 6 BV eluate was subjected to rotation under reduced pressure at 55° C. on a rotary evaporator to remove ethanol, and then freeze-dried in a vacuum freezer to obtain a purified hulless barley seed-derived polyphenol extract. The hulless barley seed-derived polyphenol extract was stored in a dark, dry, and low-temperature place for later use in the activity evaluation of controlling ulcerative colitis.

The polyphenol extracts in the following Example 1 and Comparative Examples 1, 2 and 3 were evaluated for their activities in controlling ulcerative colitis and associated colorectal cancer.

### I. Activity Evaluation for Ulcerative Colitis

### 1.1 Establishment of Ulcerative Colitis Mouse Model and Experimental Process

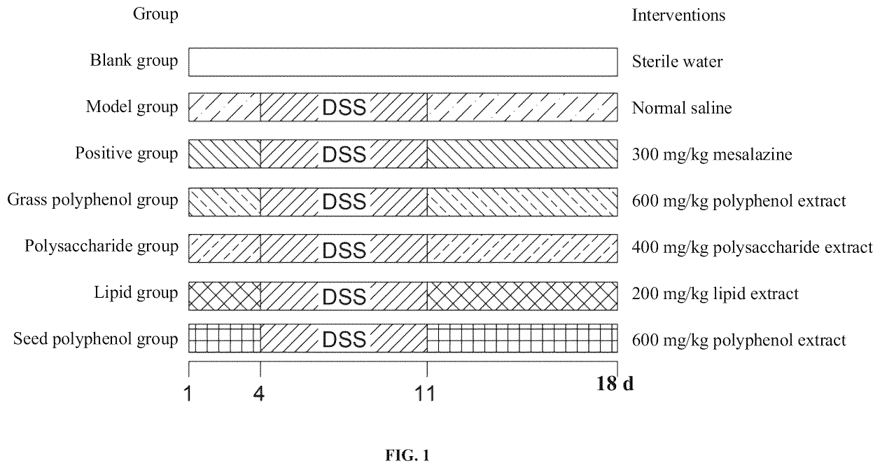

In this experiment, DSS was used to construct an acute ulcerative colitis model in mice, and a specific process is shown in FIG. 1. C57BL/6N male mice were placed in an SPF barrier environment for adaptive feeding for one week, while mice with abnormal weight were eliminated. The remaining mice were randomly divided into six groups, namely: blank group, 3% DSS+normal saline group (a model group), 3% DSS+300 mg/kg mesalazine (a positive drug group), 3% DSS+600 mg/kg hulless barley grass-derived polyphenol extract (Example 1), 3% DSS+400 mg/kg hulless barley grass-derived polysaccharide extract (Comparative Example 1), 3% DSS+200 mg/kg hulless barley grass-derived lipid extract (Comparative Example 2), and 3% DSS+600 mg/kg hulless barley seed-derived polyphenol extract (Comparative Example 3). From the first day, mice in all groups except the blank group were given drugs by gavage throughout the whole process; from the fourth day, mice in all groups (except the blank group) drank 3% DSS aqueous solution freely for one week; from the tenth day, 3% DSS was replaced with sterile drinking water for experimental mice, and the mice were allowed to recover naturally for one week; on the eighteenth day, the experiment ended and samples were collected from the mice. After the mice were anesthetized, blood was collected from their orbits. After blood collection, the liver, kidney, and spleen tissues of the mice were collected and weighed. The colon tissue from the cecum to the anus of the mice was collected and its length was measured. A 1 cm to 2 cm colon tissue from the anus was fixated in 4% paraformaldehyde solution for histopathological staining. The remaining colon tissue was quickly frozen in liquid nitrogen and then transferred to a −80° C. refrigerator to allow cryopreservation for further experiments. During the mouse modeling period, the mouse status was observed daily and the mouse weight was recorded.

### 1.2 Determination of Fecal Occult Blood and Disease Activity Index (DAI) in Mice

The fresh mouse feces were collected and the fecal occult blood was determined according to the instructions of the fecal occult blood assay kit. Specifically: 10 mg to 50 mg of feces was picked up with a toothpick and placed in a sterile culture dish. After spreading, 2 drops of pyramidon reagent were added to different positions of the feces, followed by 2 drops of oxidant. After 2 min, the color change of the feces was observed. If light blue or purple appeared, it meant that the feces contained hemoglobin or the fecal occult blood was positive. Occult blood could be divided into weakly positive, positive, and strongly positive according to color development time and color depth. The disease activity index (DAI) was a comprehensive score for the disease status of mice. The scoring criteria used in this experiment are shown in Table 1, where DAI=(weight change score+blood in stool score+stool characteristics score)/3.

TABLE 1
Scoring criteria for mouse DAI

| Score | Weight loss (%) | Occult blood/blood in stool | Normal feces |
|---|---|---|---|
| 0 | No change | Normal | Normal |
| 1 | 1-8% | Weakly positive | |
| 2 | 8-16% | Positive | Loose |
| 3 | 16-24% | Strongly positive | |
| 4 | >24% | Visible blood in stool | Watery |

### 1.3 Histopathological Staining

The colon tissue was removed from the fixative and repaired completely with a scalpel in a fume hood, placed in an embedding frame and marked, and then washed with running water for 12 h. The dehydration box was placed in the dehydrator and dehydrated in sequence. After dehydration, the tissues were made transparent in sequence, and then immersed in wax, and could be used for paraffin embedding. The wax was placed in the embedding frame, and before it solidified, the dehydrated tissue was placed in the embedding frame according to the requirements of the embedding surface and cooled at −20° C. After solidification, the wax sheet was removed from the embedding frame and trimmed. The trimmed wax blocks were sliced to a thickness of 4 μm in a paraffin microtome. The sections were placed on a slide machine and floated in 40° C. warm water to flatten the tissue, then the slides were picked up and baked in an oven at 60° C. until the water dried and the wax melted, and then stored at room temperature. The paraffin sections were dewaxed with hot water. The sections were placed in a hematoxylin staining solution and allowed to stand for 3 min to 5 min. The dye was washed off with running water, and then the sections were differentiated with a differentiation solution, rinsed with tap water, blued with a bluing solution, and rinsed with tap water. The obtained sections were dehydrated in 85% ethanol and 95% ethanol for 5 min, and then placed in an eosin staining solution for 5 min to complete dehydration and sealing. The obtained sections were sealed with neutral gum and exposed to the sun to allow image acquisition and analysis.

### 1.4 Determination of GSH, LPO, and MPO in Mouse Colon Tissue

20 mg of colon tissue from a −80° C. refrigerator was put into a 1.5 mL EP tube, then added with 6-8 grinding steel beads and 400 μL of lysis solution. The grinding module was precooled, and the 1.5 mL EP tube was placed symmetrically in the tissue grinder. The samples were ground at 18,000 rpm for 30 s and repeated 5 to 6 times until the sample solution had no obvious tissue blocks and was clear or slightly milky. The foam was removed by centrifugal force, and a portion of the obtained tissue homogenate was used for the determination of MPO. The MPO determination method was based on the myeloperoxidase (MPO) test kit method. Another part of the tissue homogenate was used for centrifugation to determine GSH and LPO, referring to the instructions of the reduced glutathione (GSH) assay kit and the instructions of the lipid peroxide (LPO) assay kit.

### 1.5 Extraction of Total RNA

Buffer RL and 50×DTT were mixed at a ratio of 1:50 to obtain a Buffer RL mixture. 20 mg of colon tissue was taken from a −80° C. refrigerator and placed in a 1.5 mL sterile EP tube, and 400 μL of lysis buffer was added. The 1.5 mL EP tubes were placed symmetrically in a tissue grinder and ground at 18,000 rpm for 30 s repeated 5 to 6 times, and pipetted repeatedly until no obvious precipitation was produced. The grinding solutions were placed symmetrically in a pre-cooled low-temperature centrifuge and centrifuged at 12,000 rpm for 5 min at 4° C. RNA was extracted according to the instructions, and then 2 μL of RNA eluate was placed in an ultra-micro spectrophotometer. The concentration and purity were determined by eluates A260 and OD260/OD280. The RNA eluate was subjected to agarose gel electrophoresis, and the integrity of the RNA was determined by the bright bands of the gel imaging system.

### 1.6 Determination of Real-Time Fluorescence Quantitative PCR Experiment

Referring to the instructions of PrimeScript™ RT reagent Kit with gDNA Eraser (Perfect Real Time), several 200 μL reaction tubes were taken, 2 μL of 5×gDNA Eraser Buffer and 1 μL of gDNA Eraser were added in sequence, an appropriate amount of extracted total RNA was added (the amount of total RNA should not exceed 1 μg), and the entire system was diluted to 10 μL with RNase Free dH₂O for the next experiment. The program of the RCR instrument was set, the reaction tube was placed at 42° C. for 2 min and then stored at 4° C. to complete the genomic DNA removal reaction. The reverse transcription reaction was conducted using the TB Green qPCR method. The amount of each reagent added to the reaction is shown in Table 2.

TABLE 2
Reverse transcription system

| Reagent | Consumption | |
|---|---|---|
| Reaction solution obtained in previous step | 10.0 | μL |
| PrimeScript RT Enzyme Mix I | 1.0 | μL |
| RT Primer Mix | 1.0 | μL |
| 5× PrimeScript Buffer 2 (for Real Time) | 4.0 | μL |
| RNase Free dH₂O | 4.0 | μL |
| Total | 20 | μL |

A program of the PCR instrument was set, the obtained reverse transcription system was reacted at 37° C. for 15 min, reacted at 85° C. for 5 s, and placed at 4° C. to terminate the reaction, and a concentration of the obtained cDNA was determined with an ultra-micro spectrophotometer. The obtained cDNA was stored at −20° C. for a long term and used in subsequent qPCR experiments. The primers used in the experiments are shown in Table 3.

TABLE 3
Names and sequences of primers during experiment

| Gene | Forward primer | Reverse primer |
|---|---|---|
| mTNF-α | CAGGCGGTGCCTATGTCTC | CGATCACCCCGAAGTTCAGTAG |
| | (SEQ ID NO: 1) | (SEQ ID NO: 2) |
| mIL-6 | TTGGTCCTTAGCCACTCCTCC | TAGTCCTTCCTACCCCAATTTCC |
| | (SEQ ID NO: 3) | (SEQ ID NO: 4) |
| mIL-1β | GCAACTGTTCCTGAACTCAACT | ATCTTTTGGGGTCCGTCAACT |
| | (SEQ ID NO: 5) | (SEQ ID NO: 6) |

Referring to the instruction manual of TB Green™ Premix Ex Taq™ II (Tli RNaseH Plus), Bulk, a 384-well plate was used for spot plate operation. The reaction system is shown in Table 4.

TABLE 4
qPCR reaction system

| Reagent | Consumption | | Final concentration | |
|---|---|---|---|---|
| TB Green Premix Ex Taq (2×) | 10 | μL | 1 | μL |
| (Tli RNaseH Plus), Bulk | | | | |
| PCR Forward Primer (10 μM) | 0.8 | μL | 0.4 | μM |
| PCR Reverse Primer (10 μM) | 0.8 | μL | 0.4 | μM |
| DNA template (<100 ng) | 2 | μL | | |
| Sterile water | 6.4 | μL | | |
| Total | 20 | μL | | |

PCR amplification was conducted using a fluorescent quantitative PCR instrument. The specific reaction conditions were as follows:
(1) 95° C. 30 s
(2) 95° C. 5 s
(3) 65° C. 30 s
(4) repeating 2 and 3 cycles 39 times
(5) 65° C. 5 s
(6) 65° C. to 95° C., with a gradient increase of 0.5° C. in each cycle.

### 1.7 Determination of Intestinal Flora in Colitic Mice

The contents of the mouse colon were collected using a 1.5 mL EP tube, immediately frozen in liquid nitrogen, and stored at −80° C. before sample delivery. 6 samples of mouse colon contents were collected from each group and placed in dry ice for 16S rRNA sequencing.

### 1.8 Implementation Effect of Example 1 and Comparative Examples 1 to 3 in Evaluating the Activity of Controlling Ulcerative Colitis

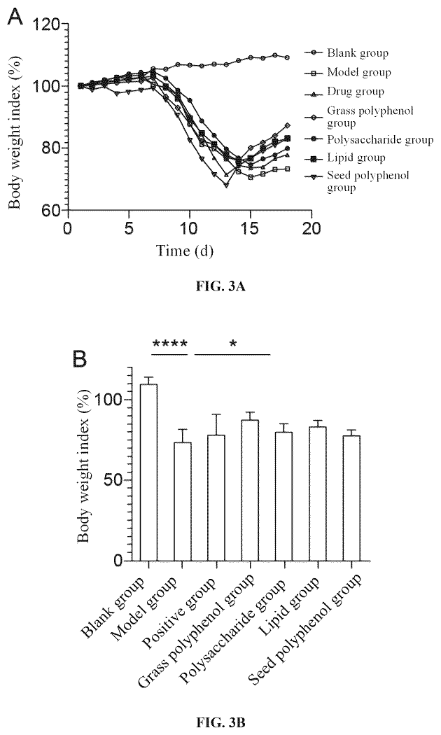

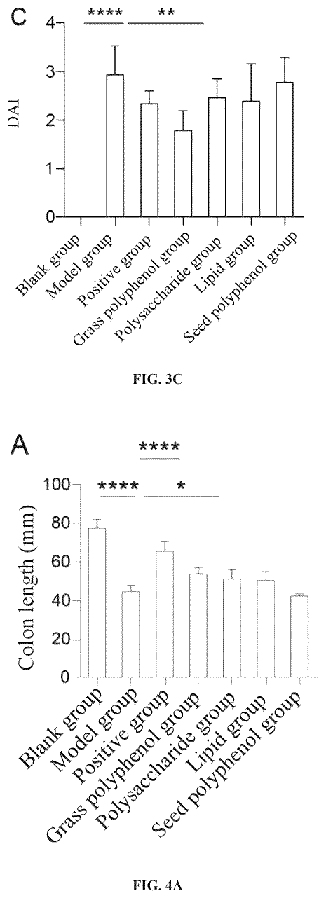

The body weight index of mice in each group is shown in FIG. 3A and FIG. 3B, where the body weight of mice in the blank group slowly increased during the entire modeling period, and the average body weight increased by the end of the experiment. Compared with the model group, Example 1, Comparative Examples 1, 2, and 3 reduced the weight loss of colitic mice to a certain extent, especially the grass polyphenol group of Example 1. In terms of the body mass index of mice, the peak body mass index of the polyphenol group in Example 1 was 75.83% higher than that of the model group. The DAI was a comprehensive score given to mice's weight loss, bloody stools, and diarrhea. As shown in FIG. 3C, in terms of the disease activity index of mice, the disease activity index of the blank group was 0, and the DAI of the model group was 2.93. Compared with the model group, all the drug-treated groups reduced the DAI of mice to some extent, among which the grass polyphenol group of Example 1 had the best effect of 1.78, which was significantly different (p<0.01). The DAI of the positive group, the grass polysaccharide group of Comparative Example 1, the grass lipid group of Comparative Example 2, and the seed polyphenol group of Comparative Example 3 were 2.33, 2.47, 2.39, and 2.77, respectively.

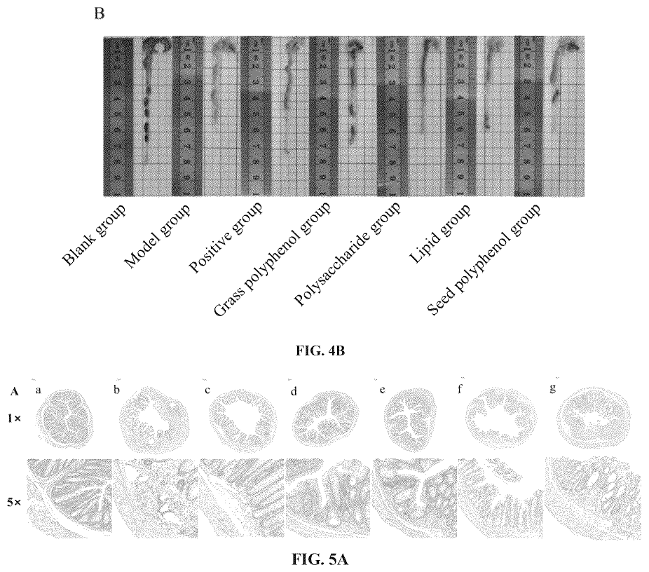

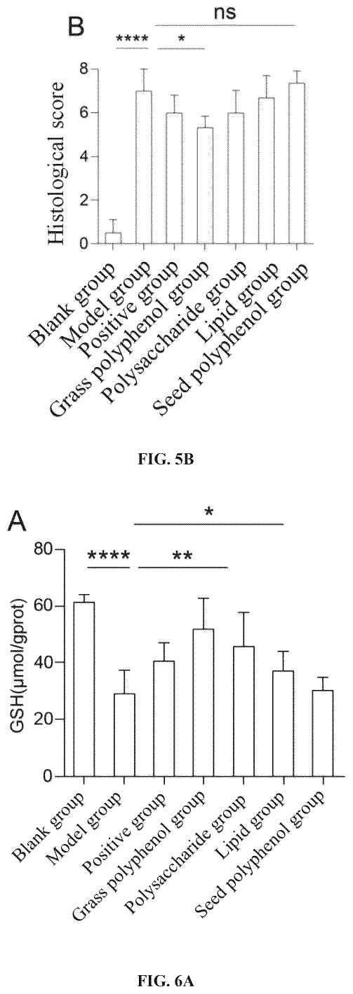

As shown in FIG. 4A, compared with the model group, the grass polyphenol group of Example 1 also significantly alleviated the shortening of the colon caused by colitis (p<0.05), and the colon of each group of mice is shown in FIG. 4B. The polysaccharide group of Comparative Example 1 and the lipid group of Comparative Example 2 also showed similar trends, but there was no significant difference in colon length between the seed polyphenol group of Comparative Example 3 and the model group, indicating that seed polyphenols could not alleviate the shortening of the colon caused by colitis. As shown in FIG. 5A, the crypt structures and goblet cells in the colon tissues of mice in the model group and the seed polyphenol group of control example 3 were mostly destroyed, a large number of inflammatory cells infiltrated, and the mucosa was severely damaged. Compared with the model group, in the grass polyphenol group of Example 1, the colon tissue crypts were relatively complete, with a small amount of inflammatory cell infiltration, and no obvious mucosal edema. According to the H&E staining results, the mouse colon was scored histopathologically according to the scoring criteria based on the degree and range of mucosal damage, and the results are shown in FIG. 5B. The histological score of the grass polyphenol group in Example 1 was lower than that of the model group, indicating that Example 1 alleviated the pathological damage of the mouse colon tissue. There was no significant difference in the histological score between the seed polyphenol group of Comparative Example 3 and the model group, indicating that Comparative Example 3 could not alleviate the pathological damage of the colon tissue of mice.

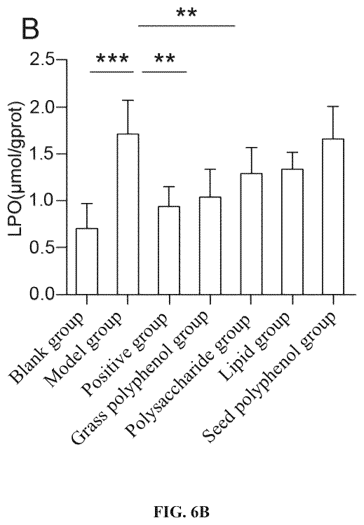

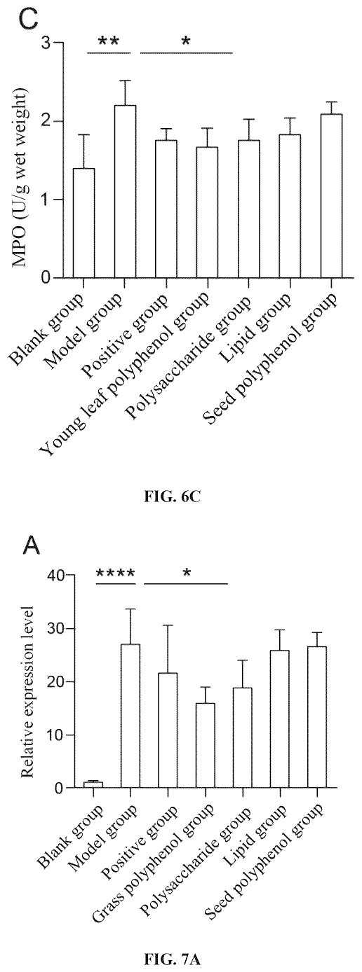

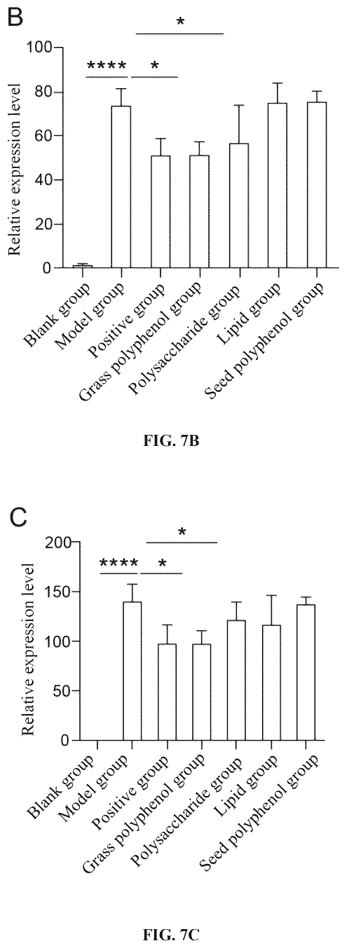

Three indicators were selected for evaluation of oxidative stress, including GSH, LPO, and MPO. As shown in FIG. 6, the grass polyphenol group of Example 1 significantly increased the GSH content and reduced the LPO content and MPO content, while there was no significant difference in LPO content and MPO content between Comparative Examples 1 and 2, indicating that Example 1 could significantly reduce the oxidative stress level of colitic mice. In terms of inflammatory factors in mouse colon tissue, FIG. 7A showed that compared with the model group, the relative mRNA expression level of TNF-α in the grass polyphenol group of Example 1 was significantly reduced (p<0.05), while there were no significant differences among the grass polysaccharide group of Comparative Example 1, the grass lipid group of Comparative Example 2, and the seed polyphenol group of Comparative Example 3. As shown in FIG. 7B and FIG. 7C, the relative mRNA expression levels of the proinflammatory cytokines IL-6 and IL-1β in the polyphenol group of Example 1 were significantly reduced (p<0.05), while there were no significant differences among the polysaccharide group of Comparative Example 1, the lipid group of Comparative Example 2, and the seed polyphenol group of Comparative Example 3. This suggested that Example 1 could reduce inflammatory response. The GSH, LPO, and MPO contents of the seed polyphenol group in Comparative Example 3 were not significantly different from those of the model group, and the oxidative stress level and inflammatory response of colitis mice could not be reduced.

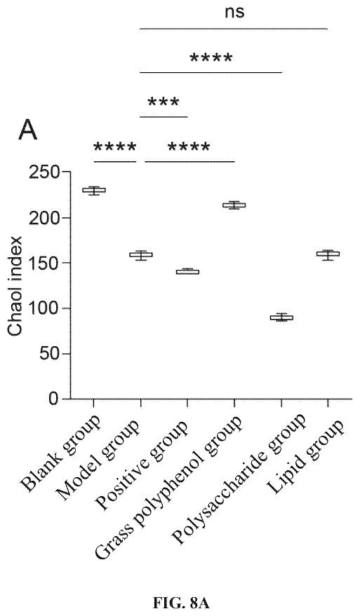

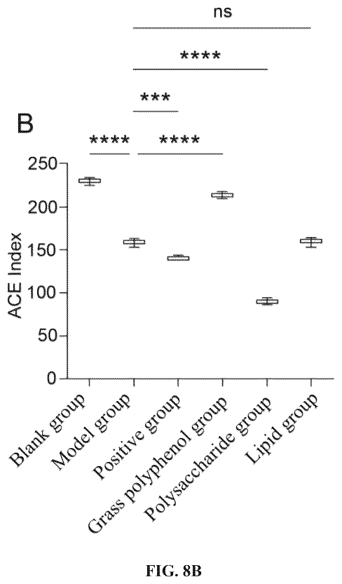

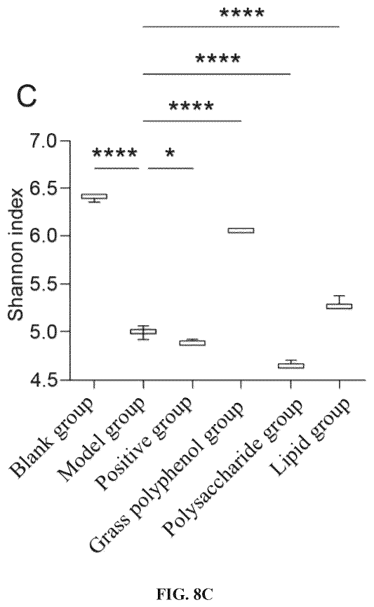

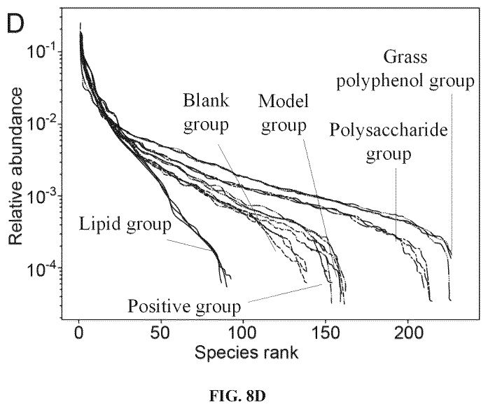

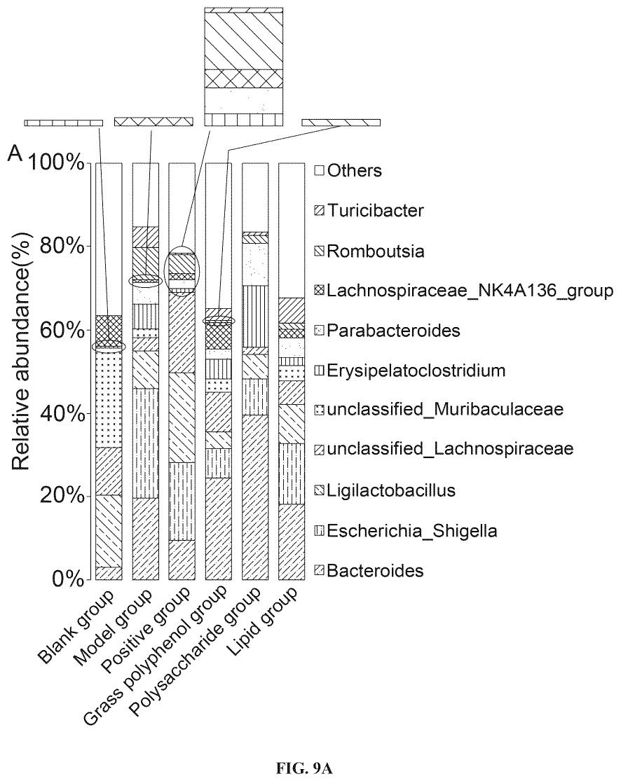

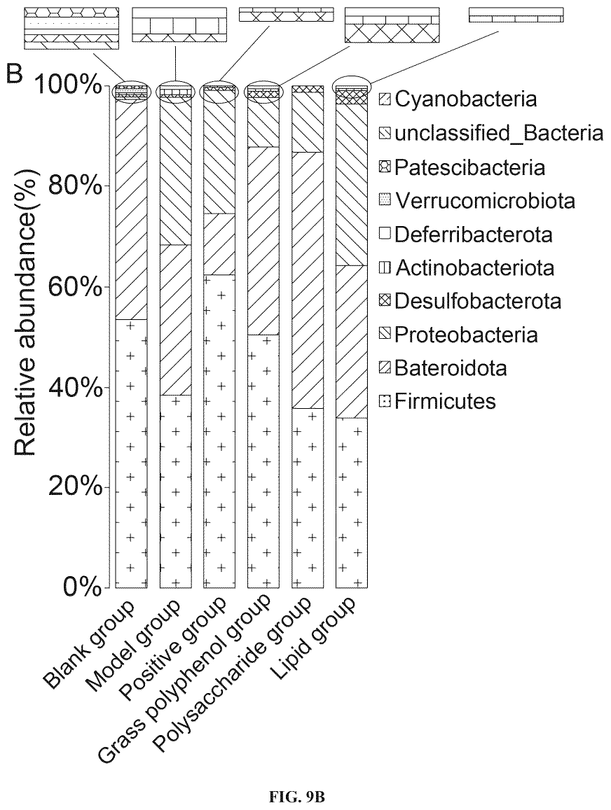

As shown in FIG. 8, the species richness and diversity of the blank group were the highest. The three indexes of the grass polyphenol group of Example 1 were significantly increased compared with the model group, indicating that polyphenol extract intervention could significantly improve the richness and diversity of the intestinal flora of enteritic mice. As shown in FIG. 8D, the intestinal flora of the mice in the grass polyphenol group of Example 1 was closest to that of the blank group in terms of richness and uniformity, indicating that the intestinal flora of the mice with colitis after intervention with the grass polyphenol group was similar to that of healthy mice. As shown in FIG. 9, the species distribution bar graph of the intestinal flora of colitis mice showed differences in the species composition of the intestinal flora of mice. At the genus level, it was seen that different groups of Shigella (Escherichia-Shigella) related to intestinal inflammation and dysbiosis appeared in all the modeling groups, among which the model group had the highest proportion, while the relative abundance of Shigella in the colon of mice in the grass polyphenol group of Example 1 was significantly downregulated. Lachnospira is a genus of bacteria that is commonly found in the intestines of healthy people and can ferment to produce acetic acid and butyric acid. The grass polyphenol group of Example 1 increased the relative abundance of Lachnospira. At the phylum level, Firmicutes and Bacteroidetes were found to be the main dominant phyla in all groups of mice. Firmicutes were important butyrate producers, while Bacteroidetes might be the main source of propionate. Compared with the model group, the grass polyphenol group of Example 1 increased the relative abundance of Firmicutes and Bacteroidetes in colitis mice.

### II. Activity Evaluation in the Ulcerative Colitis-Associated Colorectal Cancer

### 2.1 Establishment of AOM/DSS-Induced Colitis-Associated Colorectal Cancer Model in Mice

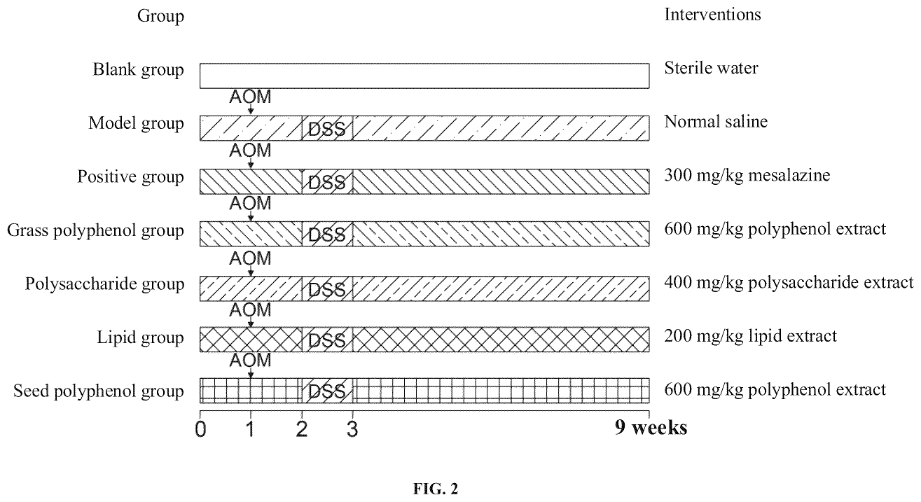

In this experiment, a mouse colitis-associated colorectal cancer model was established using AOM/DSS, and a specific process is shown in FIG. 2. C57BL/6N male mice were placed in an SPF barrier environment for adaptive feeding for one week, while mice with abnormal weight were eliminated. The remaining mice were randomly divided into six groups, namely: blank group, 3% DSS+normal saline group (a model group), 3% DSS+300 mg/kg mesalazine (a positive drug group), 3% DSS+600 mg/kg hulless barley grass-derived polyphenol crude extract (Example 1), 3% DSS+400 mg/kg hulless barley grass-derived polysaccharide crude extract (Comparative Example 1), 3% DSS+200 mg/kg hulless barley grass-derived lipid crude extract (Comparative Example 2), and 3% DSS+600 mg/kg hulless barley seed-derived polyphenol crude extract (Comparative Example 3). There were ten mice in each group. In the second week, the mice in the model group, positive group, grass polyphenol group, polysaccharide group, lipid group, and seed polyphenol group were intraperitoneally injected with 10 mg/kg AOM. From the third week, the mice drank 3% DSS aqueous solution for one week, and then the DSS aqueous solution was replaced with sterile water for the mice to drink. The experiment ended after six weeks. During the entire experiment, each group of mice was gavaged with medication according to the grouping. The conditions of mice were observed and their weights were recorded each time they were gavaged. At the end of the experiment, all mice were anesthetized, and the colon (from the anus to the cecum), liver, kidney, and spleen tissues of the mice were collected and weighed after dissection. The intestinal contents were gently rinsed with PBS and the length of the mouse colon tissue was measured. The colon was longitudinally cut open with ophthalmic scissors, and the number and size of tumors in the mouse colon were measured with a vernier caliper and recorded. A part of the whole colon of the mouse was taken for the subsequent preparation of colon Swiss rolls, fixated in 4% paraformaldehyde fixative for histopathological analysis, and the remaining tissues were quick-frozen in liquid nitrogen and then transferred to a −80° C. refrigerator for further measurement of qPCR and other experiments.

### 2.2 Preparation of Mouse Colon Swiss Roll and Histopathological Staining

The colon was taken out from the fixative in a fume hood, one end of the colon was gently clamped with ophthalmic forceps, and the entire colon was gently rolled around the forceps in one direction. Be careful not to do it too tight or too loose, otherwise the intestinal villi might fall over or become loose. The Swiss roll was prepared by piercing the edge of the rolled intestine with a fine needle to complete the fixation, and the ophthalmic forceps were removed in the center of the colon. The Swiss roll was placed in an embedding frame and labeled, followed by dehydration and wax immersion, paraffin embedding, paraffin sectioning, dewaxing to water, hematoxylin staining, eosin staining, dehydration and sealing, microscopic examination, and graphic analysis.

### 2.3 Total RNA Extraction and Fluorescence Quantitative PCR Determination

The whole colon of a mouse was divided into two major parts, namely the tumor segment and the white-side segment. 20 mg of tumor tissue and white-side tissue were taken from the same mouse for total RNA extraction and reverse transcription reaction. The specific operation was the same as above. The experimental primers used in qPCR are shown in Table 5.

TABLE 5
Name and sequence of primers

| Gene | Forward primer | Reverse primer |
|---|---|---|
| mTNF-α | CAGGCGGTGCCTATGTCTC | CGATCACCCCGAAGTTCAGTAG |
| | (SEQ ID NO: 1) | (SEQ ID NO: 2) |
| mIL-6 | TTGGTCCTTAGCCACTCCTCC | TAGTCCTTCCTACCCCAATTTCC |
| | (SEQ ID NO: 3) | (SEQ ID NO: 4) |
| mIL-1β | GCAACTGTTCCTGAACTCAACT | ATCTTTTGGGGTCCGTCAACT |
| | (SEQ ID NO: 5) | (SEQ ID NO: 6) |
| mCOX-2 | TGAGTGGGGTGATGAGCAAC | TTCAGAGGCAATGCGGTTCT |
| | (SEQ ID NO: 7) | (SEQ ID NO: 8) |
| mEGFR | GGAGAGAATCCCTTTGGAGAAC | CCAGTTCTGTTTGTCCCATAGT |
| | (SEQ ID NO: 9) | (SEQ ID NO: 10) |
| mINOS | GTTCTCAGCCCAACAATACAAGA | GTGGACGGGTCGATGTCAC |
| | (SEQ ID NO: 11) | (SEQ ID NO: 12) |
| mcatenin | GGCAACCCTGAGGAAGAAGA | GGGATGAGCAGCGTCAAACT |
| | (SEQ ID NO: 13) | (SEQ ID NO: 14) |
| mMyc | GGAACGTCAGAGGAGGAACG | TGCTCGTCTGCTTGAATGGA |
| | (SEQ ID NO: 15) | (SEQ ID NO: 16) |
| mCyclinD1 | TCCTGCTACCGCACAACG | GACCAGCCTCTTCCTCCAC |
| | (SEQ ID NO: 17) | (SEQ ID NO: 18) |
| mβ-actin | GGCTGTATTCCCCTCCATCG | CCAGTTGGTAACAATGCCATGT |
| | (SEQ ID NO: 19) | (SEQ ID NO: 20) |

### 2.4 Determination of Intestinal Flora in Colorectal Cancer Mice

The mouse feces was collected using a 1.5 mL EP tube, immediately frozen in liquid nitrogen, and stored at −80° C. before sample delivery. 6 samples of mouse colon contents were collected from each group and placed in dry ice for 16S rRNA sequencing by Qingdao Biomarker Technologies Co., LTD.

### 2.5 Statistical Analysis

The experimental data were plotted using GraphPad Prism9.0, and statistical analysis was conducted using one-way ANOVA, where “ns” indicated no significant difference, “*” indicated p<0.05, “**” indicated p<0.01, “***” indicated p<0.001, and “****” indicated p<0.0001. The relative expression of mRNA in qPCR was calculated using the 2-4CT method.

### 2.6 Implementation Effect of Example 1, Comparative Examples 1, 2, and 3 in Evaluating the Activity of Controlling Colorectal Cancer

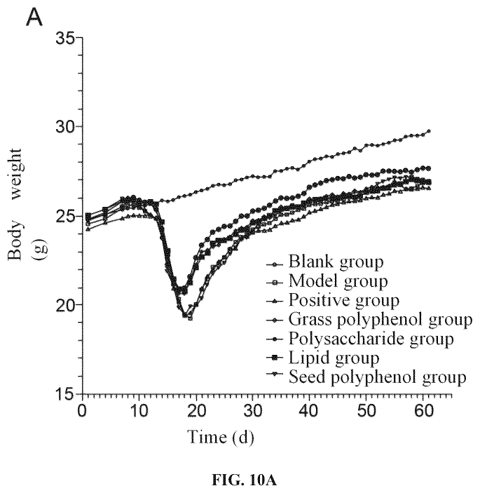

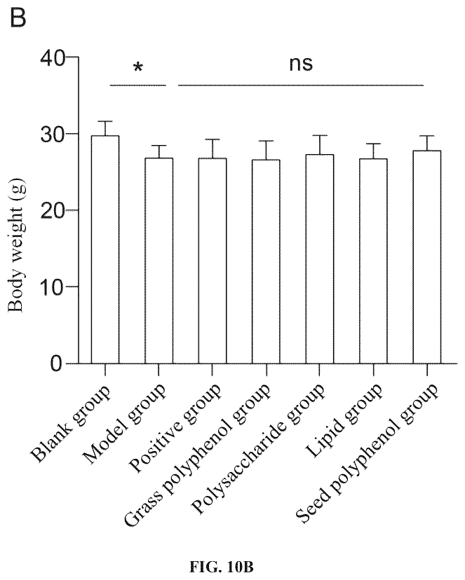

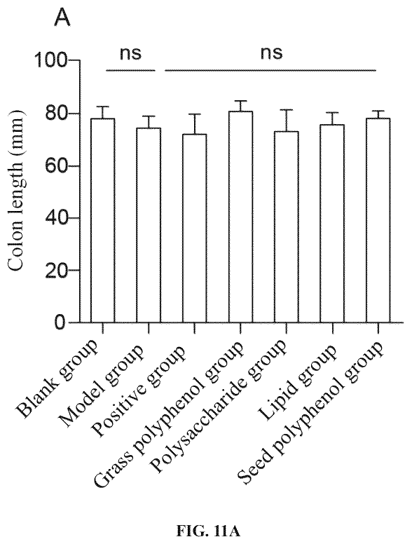

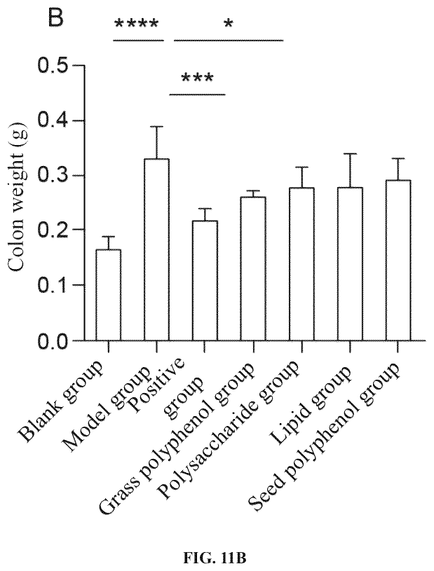

As shown in FIG. 10, the mice experienced different degrees of weight loss while drinking DSS water. The weight loss of mice in the model group was larger, while the weight loss of mice in the grass polyphenol group of Example 1, the grass polysaccharide group of Comparative Example 1, the grass lipid group of Comparative Example 2, and the seed polyphenol group of Comparative Example 3 was smaller. However, as the experiment progressed, the activity status of the mice gradually deteriorated, and in the middle and late stages of the experiment, occult blood appeared in the feces of the mice in the model group. In the later stage of the experiment, the mice developed loose hair, depression, bloody stools, and rectal prolapse. When the experiment was over, the body weight of the mice in the model group, the positive drug group, the grass polyphenol group of Example 1, the grass polysaccharide group of Comparative Example 1, the grass lipid group of Comparative Example 2, and the seed polyphenol group of Comparative Example 3 all showed a significant decrease. The results confirmed that tumor growth could lead to an increase in colon weight. As shown in FIG. 11B, the colon weight of the model group mice was significantly increased compared with that of the blank group mice. The grass polyphenol group in Example 1 significantly reduced the increase in colon weight caused by modeling. The mice in the grass polysaccharide group, lipid group, and seed polyphenol group also showed similar trends. Colon weight per unit length is a ratio of colon weight to colon length and can reduce the effect of colon length on colon weight. As shown in FIG. 11C, since there was no significant difference in colon length among the mice in each group, the colon weight per unit length and the colon weight of each group of mice showed a consistent trend. The results showed that the positive group and the polyphenol group in Example 1 effectively reduced the increase in tumor weight caused by modeling.

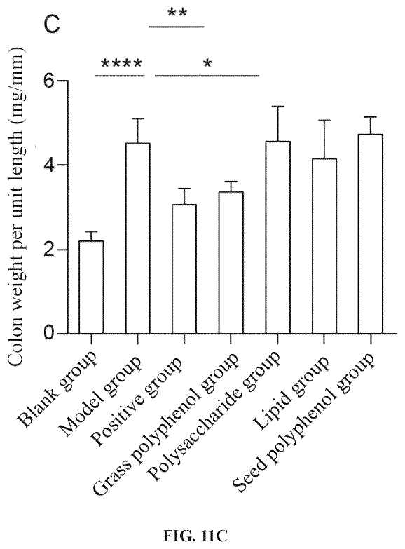

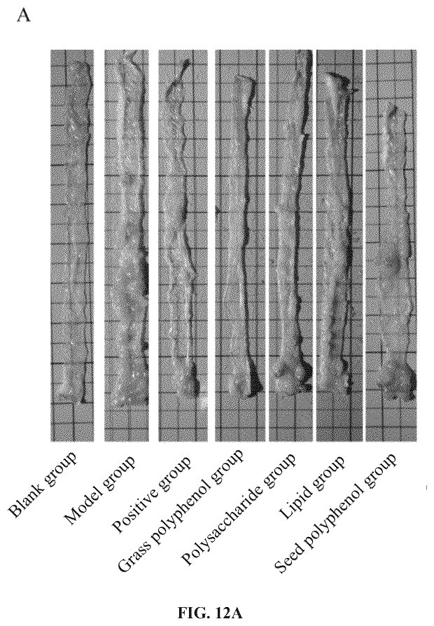

The most intuitive manifestation of ulcerative colitis-associated carcinogenesis was the appearance of tumors of different sizes in multiple locations in the colon, mostly at the anus and in the middle of the colon, as shown in FIG. 12A, which represented typical colon images of mice in different treatment groups. The number of tumor nodules and the proportion of tumors of different grades are the most intuitive indicators reflecting the number and size of mouse colon tumors. As shown in FIG. 12B and FIG. 12C, the blank group had 0 tumor nodule, and the number of tumors in the grass polyphenol group of Example 1 decreased significantly compared with the model group. From the proportion of tumors of different grades, it was seen that compared with the model group, the proportion of tumors less than 2 mm in the positive group and the grass polyphenol group of Example 1 increased significantly, while the proportion of tumors of 2 mm to 3 mm and above 3 mm decreased. Therefore, it was concluded that hulless barley polyphenol extract and mesalazine effectively inhibited the development of ulcerative colitis-associated carcinogenesis. There were no significant differences in tumor nodules and the proportion of tumors larger than 3 mm between the mice in the polysaccharide group of Comparative Example 1, the lipid group of Comparative Example 2, and the seed polyphenol group of Comparative Example 3, and the model group, indicating that hulless barley grass-derived polysaccharide and lipid extracts and hulless barley seed-derived polyphenol could not inhibit the development of tumors.

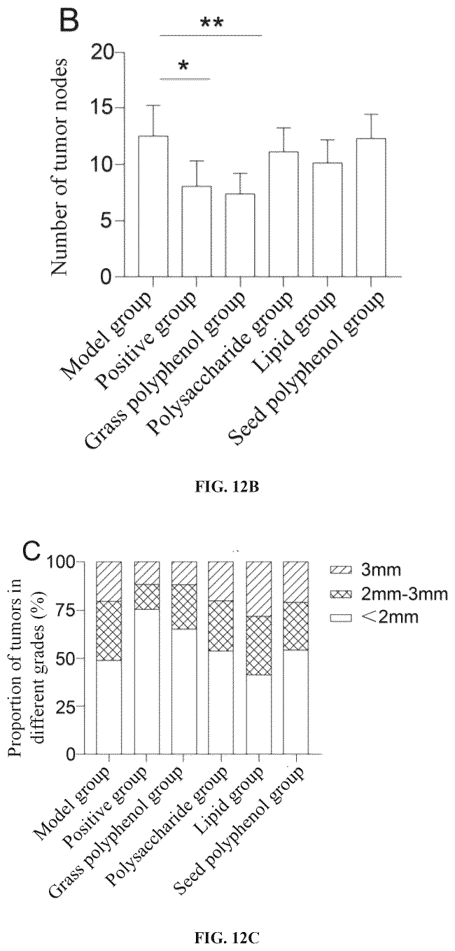

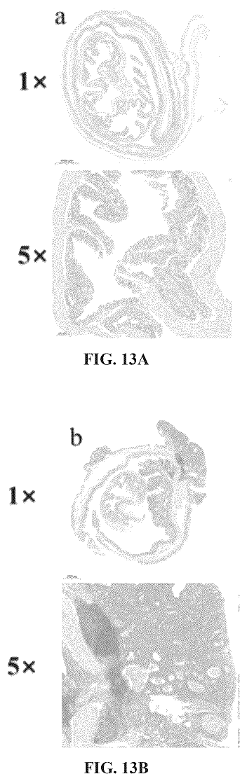

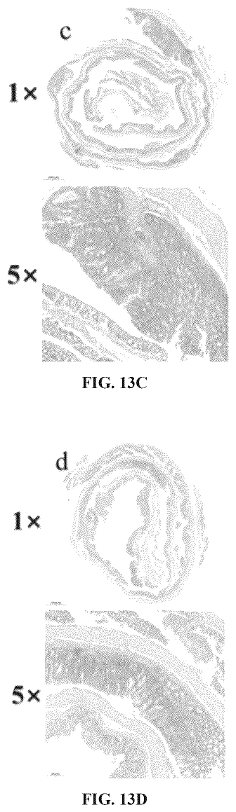

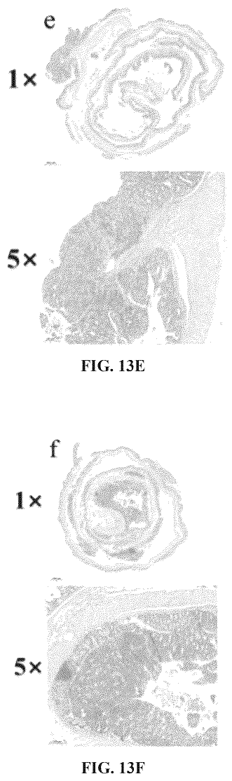

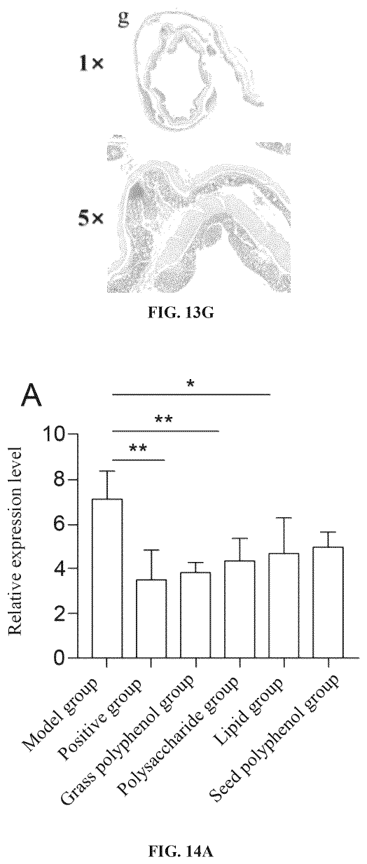

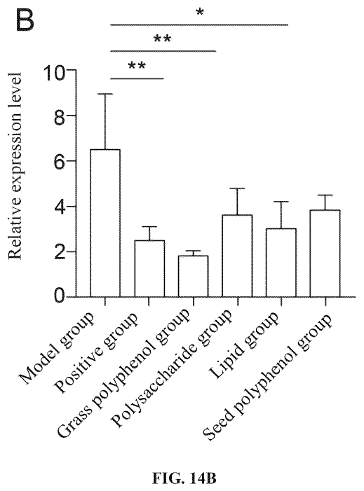

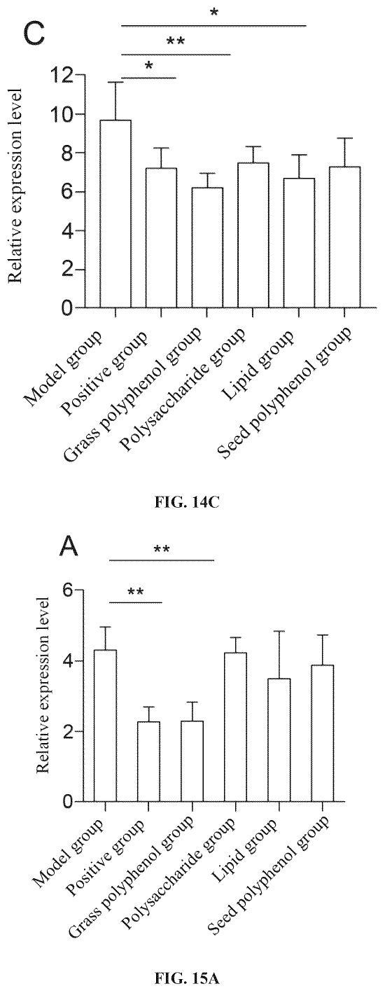

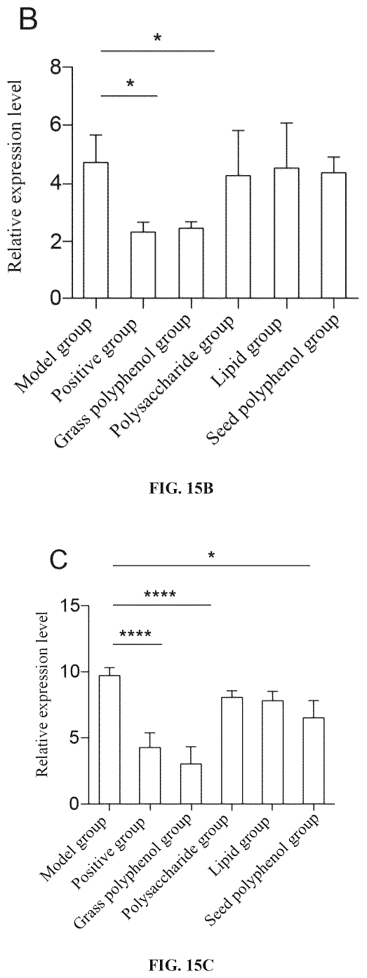

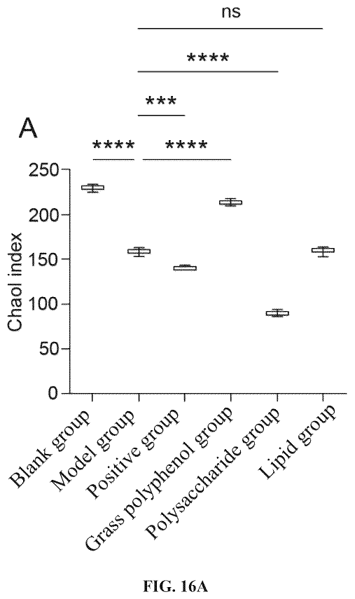

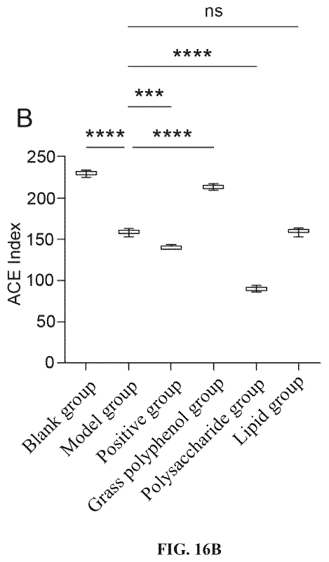

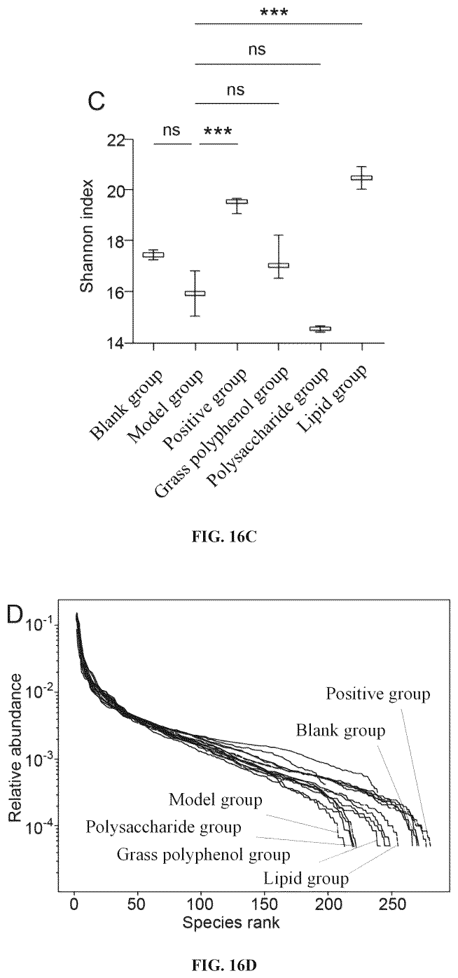

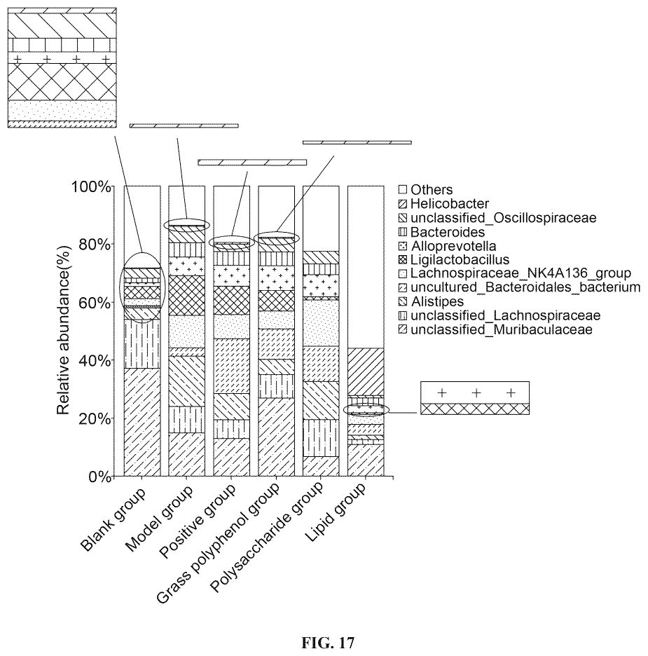

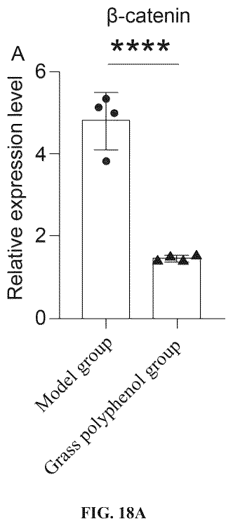

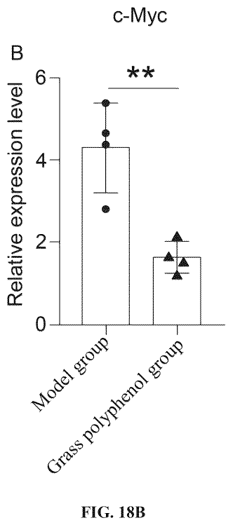

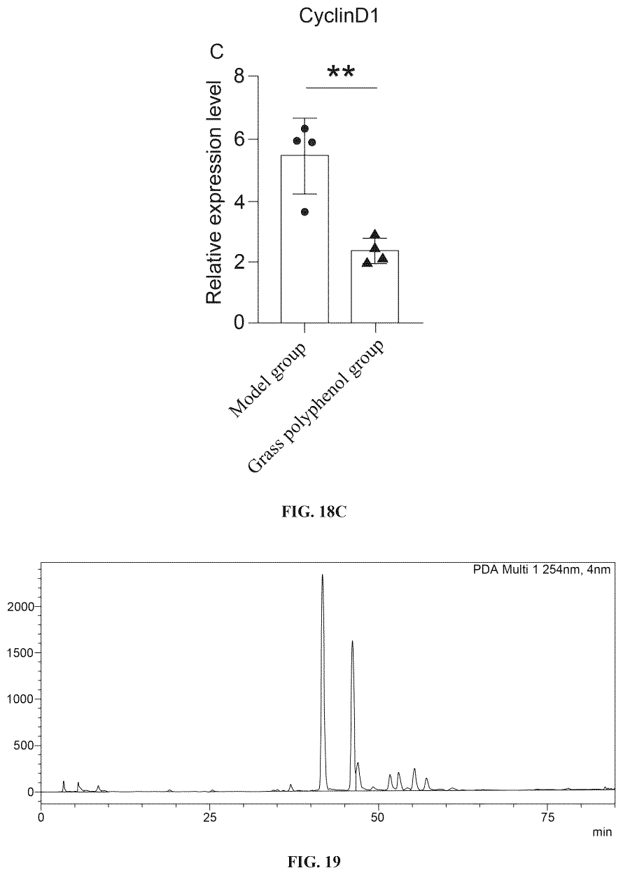

The results of pathological sections of the mouse colon group are shown in FIG. 13. The colon tissue of the mice in the model group was significantly thickened, and the tumors in the colon tissue were large and numerous. Compared with the model group, the colon thickness of the mice in the grass polyphenol group of Example 1 was reduced, and the tumors in the colon tissue were small and few in quantity. The positive group also showed a similar trend. The results showed that hulless barley grass-derived polyphenol extract and mesalazine had the effect of preventing carcinogenesis of ulcerative colitis-associated colorectal cancer in mice. As shown in FIG. 14, compared with the model group, the relative expression levels of TNF-α, IL-6, and IL-1β mRNA in the tumor tissue of mice from the positive drug group and the grass polyphenol group of Example 1 were significantly reduced, indicating that the hulless barley grass-derived polyphenol extract and mesalazine in the drug group could inhibit chronic inflammation in the development of colitis-associated colorectal cancer. INOS and COX-2 are both induced enzymes that can be activated by a variety of proinflammatory cytokines, catalyzing the production of high concentrations of nitric oxide, thereby increasing the level of ROS in vivo and causing DNA damage. EGFR is widely considered to be a tumor promoter, with increased expression or activation in many colon premalignant lesions and tumors. As shown in FIG. 15, compared with the model group, the expression levels of COX-2, EGFR, and INOS genes in the tumor tissues of mice in the positive drug group and the grass polyphenol group of Example 1 were significantly downregulated. As shown in FIG. 16, the three diversity indices of the grass polyphenol group in Example 1 were significantly increased compared with the model group. The intervention of hulless barley grass-derived polyphenol extract could significantly improve the richness and diversity of the intestinal flora in colon cancer mice. The richness and uniformity of the intestinal flora in the positive drug group were closest to those of the blank group, followed by the grass polyphenol group in Example 1. This indicated that mesalazine was a desirable therapeutic agent for the intestinal flora disorder in mice with colitis-associated colorectal cancer, and the grass polyphenol group also had a certain improvement in the reduced diversity of intestinal flora caused by colorectal cancer modeling. The species distribution bar graph of the intestinal flora of colorectal cancer mice showed differences in the species composition of the intestinal flora of mice. As shown in FIG. 17, the grass polyphenol group of Example 1 significantly increased the relative abundance of Bacteroidetes in the intestine and effectively delayed the decrease in the relative abundance of Lactobacillus. Further research on the mechanism of action showed that the Wnt signaling pathway was one of the most representative signaling pathways in colorectal cancer and played an important role in the occurrence of colorectal cancer. As shown in FIG. 18, compared with the model group, the expression level of β-catenin, a key component of the Wnt signaling pathway, in the grass polyphenol group of Example 1 decreased, and the relative expression levels of downstream target genes C-Myc and CyclinD1 also decreased significantly, indicating that the hulless barley grass-derived polyphenol extract could inhibit the formation of colorectal cancer tumors by inhibiting the Wnt signaling pathway.

The specific embodiments of the present disclosure are described above. It should be understood that the present disclosure is not limited to the above specific implementations, and a person skilled in the art can make various variations or modifications within the scope of the claims without affecting the essence of the present disclosure.

What is claimed is:

1. A method for preventing and/or treating ulcerative colitis and associated colorectal cancer thereof, comprising administering a medicament to a subject in need thereof, wherein the medicament comprises hulless barley grass-derived polyphenol extract and wherein a method for preparing the hulless barley grass-derived polyphenol extract comprises the following steps:
S1, subjecting a hulless barley grass whole powder to alcohol extraction, evaporation, and freeze-drying to obtain a crude polyphenol extract; and
S2, separating the crude polyphenol extract using a chromatography column loaded with an activated saturated wet resin, and then purifying the crude polyphenol extract to obtain the hulless barley grass-derived polyphenol extract.

2. The method according to claim 1, wherein the alcohol extraction in step S1 comprises: mixing the hulless barley grass whole powder with a methanol solution to allow an ultrasonic treatment and centrifugation in sequence to obtain a supernatant.

3. The method according to claim 2, wherein the methanol solution has a mass percentage of 70% to 90%; and the hulless barley grass whole powder and the methanol solution are at a material-to-liquid ratio of 1 g:(20-25) mL.

4. The method according to claim 2, wherein the ultrasonic treatment is conducted under a power of 480 W to 560 W at 40° C. to 50° C. for 30 min to 60 min.

5. The method according to claim 1, wherein a preparation process of the activated saturated wet resin in step S2 comprises: subjecting a macroporous resin to immersing in an ethanol solution, washing with deionized water, immersing in a hydrochloric acid solution and a sodium hydroxide solution, and then adjusting a pH value in sequence.

6. The method according to claim 5, wherein the macroporous resin comprises an AB-8 macroporous resin.

7. The method according to claim 5, wherein the macroporous resin is immersed in an ethanol solution with a mass percentage of 80% to 95% for 15 h to 28 h.

8. The method according to claim 5, wherein a total volume of the hydrochloric acid solution and the sodium hydroxide solution is 3 to 5 times a volume of the macroporous resin; and the macroporous resin is immersed in a hydrochloric acid solution with a mass percentage of 3% to 5% and a sodium hydroxide solution with a mass percentage of 2% to 5% for 2 h to 6 h.

9. The method according to claim 1, wherein the separating in step S2 comprises dissolving the crude polyphenol extract into a sample solution, loading the sample solution onto the chromatography column, removing impurities, and then eluting the hulless barley grass-derived polyphenol extract to obtain an eluate.

10. The method according to claim 9, wherein the sample solution has a concentration of 2 mg/mL to 3 mg/mL, 4 BV of the sample solution is loaded at a flow rate of 2 BV/min to 3 BV/min, the impurities are removed with 4 BV of deionized water, and then the hulless barley grass-derived polyphenol extract is eluted with 70% ethanol at a flow rate of 2.5 BV/min to 3 BV/min.
